(19) United States
(12) Patent Application Publication
(10) Pub. No.: US 2014/0206897 A1
(43) Pub. Date: Jul. 24, 2014

(54) METHOD FOR MAKING METHYL METHACRYLATE FROM PROPIONALDEHYDE AND FORMALDEHYDE VIA OXIDATIVE ESTERIFICATION

(71) Applicant: Saudi Basic industries Corporation, Riyadh (SA)

(72) Inventors: James Malcolm Allman, Sugar Land, TX (US); James Kauffman, Katy, TX (US)

(73) Assignee: Saudi Basic industries Corporation, Riyadh (SA)

(21) Appl. No.: 14/159,032

(22) Filed: Jan. 20, 2014

Related U.S. Application Data
(60) Provisional application No. 61/755,260, filed on Jan. 22, 2013.

(51) Int. Cl. C07C 67/44

(52) U.S. Cl. CPC C07C 67/44
USPC 560/210

(57) ABSTRACT

A process for forming methyl methacrylate can comprise: reacting ethylene, carbon monoxide, and hydrogen, in the presence of a first catalyst comprising a metal carbonyl; removing a first reaction product comprising propionaldehyde; reacting the first reaction product with formaldehyde; removing a second reaction product comprising methacrolein; reacting the second reaction product with oxygen and methanol in the presence of a second catalyst to form a third reaction product comprising methyl methacrylate. Another process for forming methyl methacrylate can comprising: reacting ethylene with carbon monoxide to form propionaldehyde; reacting the propionaldehyde with formaldehyde to form methacrolein; and reacting the methacrolein with methanol and oxygen to form the methyl methacrylate.

### CROSS-REFERENCE TO RELATED APPLICATION

The present application claims the benefit of U.S. Provisional Application Ser. No. 61/755,260, filed Jan. 22, 2013, the contents of which is hereby incorporated in its entirety by reference.

### BACKGROUND OF THE INVENTION

This disclosure relates to methods of manufacture of methyl methacrylate.

Methyl methacrylates are useful in the manufacture of articles and components for a wide range of applications, from medical technologies to transparent glass substitutes. Because of their broad use, it is desirable to manufacture methyl methacrylates efficiently and economically. However, the existing processes suffer from drawbacks, including complex processing and expensive materials.

There accordingly remains a need in the art for producing methyl methacrylate while reducing the amount of processing required and improving economic competitiveness.

### SUMMARY OF THE INVENTION

Disclosed herein are processes for making methyl methacrylate.

In an embodiment, a process for forming methyl methacrylate can comprise: reacting ethylene, carbon monoxide, and hydrogen, in the presence of a first catalyst comprising a metal carbonyl; removing a first reaction product comprising propionaldehyde; reacting the first reaction product with formaldehyde; removing a second reaction product comprising methacrolein; reacting the second reaction product with oxygen and methanol in the presence of a second catalyst to form a third reaction product comprising methyl methacrylate.

In another embodiment, another process for forming methyl methacrylate can comprise: reacting ethylene with carbon monoxide to form propionaldehyde; reacting the propionaldehyde with formaldehyde to form methacrolein; and reacting the methacrolein with methanol and oxygen to form the methyl methacrylate.

The above described and other features are exemplified by the following detailed description.

### BRIEF DESCRIPTION OF THE DRAWINGS

Refer now to the FIGURE, which is exemplary not limiting.

FIG. 1 is a schematic of an embodiment of the process disclosed herein.

### DETAILED DESCRIPTION OF THE INVENTION

There are several methods of manufacturing methyl methacrylates. The routes include: acetone cyanohydrin, isobutylene oxidation, propionaldehyde formylation, propionic acid formylation, and methyl propionate formylation.

In the acetone cyanohydrin route (1) below, acetone is reacted with hydrogen cyanide to form acetone cyanohydrin. The cyanohydrin is then hydrolyzed and dehydrated by various means, usually sulfuric acid, and then reacted with methanol or a methanol derivative to form methyl methacrylate (MMA).

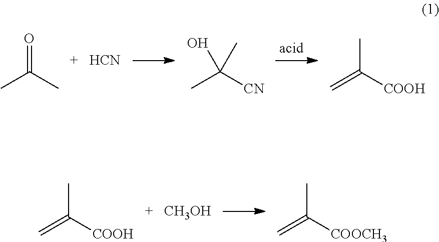

In the isobutylene oxidation process (2), illustrated below, isobutylene (or t-butyl alcohol) is oxidized via vapor-phase oxidation to methacrolein, and the methacrolein is subsequently oxidized to methacrylic acid, also via vapor-phase oxidation. The methacrylic acid is subsequently esterified with methanol to form MMA.

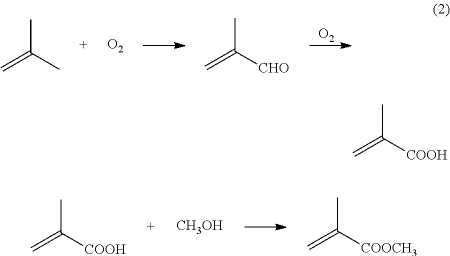

In a modification to the isobutylene oxidation process, illustrated below (3) the last two steps are combined, such that oxidation of methacrolein and esterification with methanol take place in the same reactor, in a reaction known as “oxidative esterification.” Not only does this improvement eliminate reaction and purification equipment by combining steps, it specifically gets rid of the step of oxidizing methacrolein to methacrylic acid, which has several challenges in catalyst manufacture and performance.

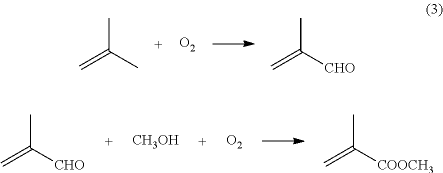

In the propionaldehyde formylation process, illustrated below (4), ethylene is first hydroformylated with CO and hydrogen to form propionaldehyde. Next, the propionaldehyde is reacted with formaldehyde in an aldol condensation to form methacrolein. The methacrolein is then oxidized to methacrylic acid, typically in a liquid-phase process. Finally, the methacrylic acid is esterified with methanol to form MMA.

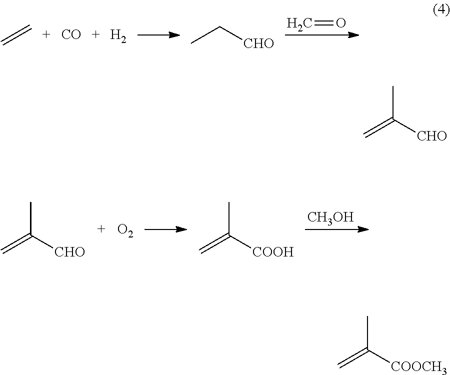

In the propionic acid formylation process, illustrated below (5), ethylene is first carbonylated to form propionic acid. The propionic acid is then reacted with formaldehyde to form methacrylic acid. The resulting methacrylic acid is then esterified with methanol to form MMA.

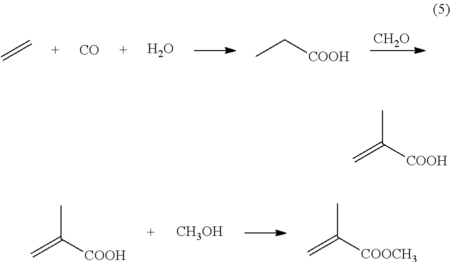

In the methyl propionate formylation process, illustrated below (6), ethylene is carbonylated in the presence of methanol to form methyl propionate. The methyl propionate is then reacted with formaldehyde to form MMA directly.

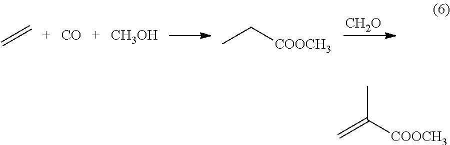

The six processes discussed above can be classified cleanly into three classes. The first class is the acetone cyanohydrin process, illustrated as route (1) above. The second class is the isobutylene oxidation process, illustrated as routes (2)-(3) above. The final class includes the last three processes, routes (4)-(6) above, which share the features of ethylene carbonylation and formylation (reaction with formaldehyde) of the resulting C₃ compound. This last class can be referred to as the “ethylene-based” processes.

Economically, the first class is the worst, as it requires expensive hydrogen cyanide, and the process normally uses sulfuric acid, which must be cleaned up at high cost. The second class is the next least desirable from an economic perspective, as it uses reasonably cheap isobutylene, and does not require expensive processing of any reagents. The final class is the most economical, as two of the four carbons in the methacrylic acid structure come from inexpensive C₁ compounds (CO and formaldehyde), while the other two carbons come from ethylene, which is reasonably priced. Also, compared with the other classes, the amount of processing is not excessive (with the possible exception of the propionaldehyde formylation process).

Among the ethylene-based processes, routes (4)-(6) above, it is expected that the methyl propionate formylation process is the most economical, as it involves the least processing. Propionic acid formylation is the next most economical, followed by propionaldehyde formylation, which involves the most processing.

The problem with both of the two least expensive ethylene-based processes, methyl propionate formylation (6) and propionic acid formylation (5), is that these formylation reactions are not favored by nature, and thus must be strongly assisted by catalyst, conditions, etc. The result is awkward and expensive processing, involving catalyst processing, high formaldehyde concentrations, large recycles, etc. By contrast, the most expensive process in this class, propionaldehyde formylation (4), uses a formylation reaction that is highly favored by nature, which is referred to as “aldol condensation.” Since this formylation produces methacrolein rather than methacrylic acid or MMA directly, it involves more subsequent processing, making it economically less desirable.

Without being bound by theory, it is believed that the favorable results obtained herein, e.g., an economical and efficient process for manufacturing methyl methacrylate, are achieved through modification of the propionaldehyde formylation process to include the oxidation and esterification of methacrolein.

The process can embody a combination of oxidation and esterification of methacrolein, which is ideally suited for use in the propionaldehyde formylation process. The combination is believed to work better with propionaldehyde formylation than with isobutylene oxidation, as the latter involves a vapor-phase oxidation with associated inert gas, high temperatures, etc. By combining propionaldehyde formylation with oxidative esterification, a process can be constructed that retains the raw material advantages of the ethylene-based processes, retains the use of aldol condensation, and does not involve too much processing.

“Methyl Methacrylate” means compositions having structural units of formula (7):

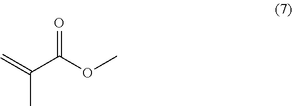

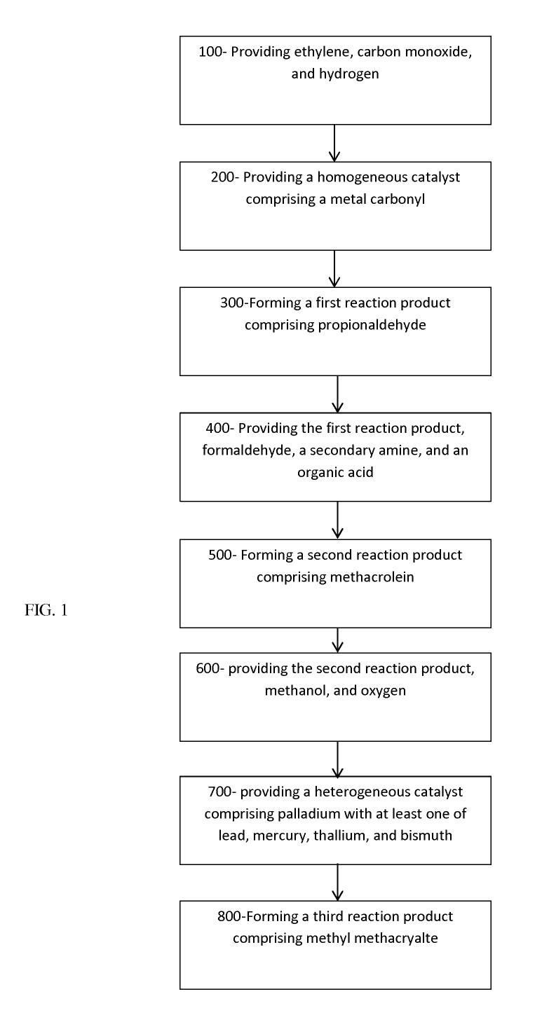

FIG. 1 illustrates a process for the manufacture of methyl methacrylate. The process can include Step 100: providing ethylene, carbon monoxide, and hydrogen, in a solvent, in the presence of a homogeneous catalyst (Step 200), e.g., a metal carbonyl, for example, a cobalt carbonyl or rhodium carbonyl, in the presence of ligands such as triphenyl phosphine or substituted triphenyl phosphine to form a first reaction product. The first reaction product comprises propionaldehyde (Step 300). Subsequently, the first reaction product can be reacted with formaldehyde, a secondary amine, and an organic acid (Step 400) to form a second reaction product comprising methacrolein (Step 500). The methacrolein is reacted with methanol and oxygen (Step 600) in the presence of a heterogeneous catalyst (Step 700) to form a third reaction product comprising methyl methacrylate (Step 800).

Step 100 comprises providing ethylene, carbon monoxide, and hydrogen, in a solvent. The reactants can be provided in a reactor, e.g., a pressurized stirred tank reactor. The reaction conditions can include total pressures of 100 psig (pounds per square inch) to 3000 psig or 790 kPa (kiloPascal) to 20785 kPa, and temperatures of 50° C. (degrees Celsius) to 200° C. Specifically, pressures can be 500 psig to 1500 psig or 3548 kPa to 10443 kPa, and temperatures can be 100° C. to 150° C.

A homogeneous catalyst is then provided in the reactor in step 200. The homogeneous catalyst can include a metal carbonyl. For example, the catalyst can comprise a cobalt carbonyl or rhodium carbonyl, in the presence of ligands such as triphenyl phosphine or substituted triphenyl phosphine. The homogeneous catalyst can include combinations of cobalt, rhodium, iridium, and ruthenium with biphyllic ligands comprising phosphorus, arsenic, and bismuth.

Step 300 includes forming a first reaction product from the reactants and catalyst described above. The first reaction product can comprise propionaldehyde. The propionaldehyde can be separated from additional reaction products through filtering, washing, distillation, or combination thereof.

The second reaction product comprising propionaldehyde is provided to a reactor, e.g., a stirred tank reactor, with formaldehyde and additional reactants in Step 400. The formaldehyde can be provided in a range of stoichiometric amounts of aqueous formaldehyde. For example, the molar ratio of formaldehyde to propionaldehyde can be 1:1 to 1.5:1. The additional reactants can include a secondary amine, in molar ratios of 0.005:1 to 0.1:1 of the propionaldehyde. For example, the secondary amine can include di-2-ethyl hexylamine, diphenylamine, dicyclohexylamine, dipropylamine, methylbutylamine, ethylbutylamine, diisooctylamine, piperidine, pyrrolidine, piperazine, morpholine, and combinations thereof. In addition, the additional reactants can include an organic acid of up to 8 carbon atoms at a molar ratio of 0.002:1 to 0.05:1 of the propionaldehyde. Exemplary organic acids include formic acid, oxalic acid, maleic acid, acetylene, dicarboxylic acid, acetic acid, propionic acid, n- or i-butanoic acid, malonic acid, glutaric acid, succinic acid, tartaric acid, adipic acid, hydroxy succinic acid, salicylic acid, 2-ethylhexanoic acid, and combinations thereof.

The reaction conditions can be at a temperature of 70° C. to 120° C., under an autogeneous pressure of approximately 1.0 to 3.0 atmospheres or 101.3 kPa to 303.9 kPa. More specifically, the reaction conditions can be 80° C. to 100° C., and the reaction pressure can be 1.5 atmospheres to 2.5 atmospheres, or 151.9 kPa to 253.3 kPa. Step 500 includes forming a second reaction product from the reactants described above. The second reaction product comprises methacrolein. The methacrolein can be separated from other reaction products and materials through distillation, e.g., fractional distillation.

The second reaction product is provided to a reactor, e.g., a low pressure stirred tank reactor, with methanol and oxygen in Step 600. The oxygen can be bubbled through the reactor. A catalyst is simultaneously provided in Step 700. The catalyst can be a heterogeneous catalyst. For example, the catalyst can comprise palladium with at least one of lead, mercury, thallium, gold, copper, silver, cadmium, zinc, indium, tin, antimony, and bismuth, and preferably, at least one of lead, mercury, thallium, and bismuth. Specifically, the catalyst can comprise palladium and lead. More specifically, the catalyst can comprise palladium and lead in a molecular ratio of 3:1. The catalyst can be suspended in the reaction mixture. The catalyst can be supported on calcium carbonate.

The reaction conditions can include a temperature of 50° C.-100° C. In addition, the reaction can include a pressure of 1.0 atmosphere or 101.3 kPa. The reaction product formed in Step 800 comprises methyl methacrylate. The conversion of methacrolein to methyl methacrylate is greater than 95%. Specifically, the conversion of methacrolein to methyl methacrylate is greater than 97%. The methyl methacrylate may be separated from the catalyst through filtering, and separated from by-products and other materials through distillation.

The entire process may be illustrated as follows (8):

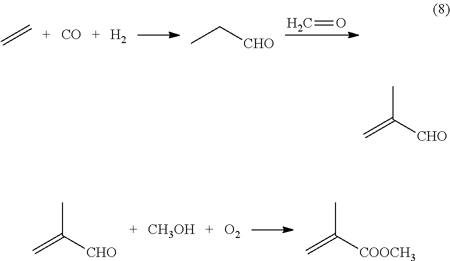

Propionaldehyde is manufactured by the hydroformylation (e.g., reaction with a 1:1 mixture of hydrogen and carbon monoxide) of ethylene, typically in the presence of a solvent and a homogeneous catalyst. The catalyst is a complex of a metal active in hydroformylation, typically cobalt, rhodium, or nickel. Various ligands are used in the catalyst complex, typically phosphite ligands.

Methacrolein can be made from propionaldehyde and formaldehyde in a reaction known as “aldol condensation.” Aldol condensation is an extremely well-known reaction, discussed in most undergraduate organic chemistry textbooks (something which is typically not true of hydroformylation, and certainly not true of oxidative esterification).

Aldol condensations can occur under a variety of conditions, including weak acid, strong base, with various catalysts, etc. However, a preferred way to run this particular aldol condensation is with a catalyst system comprising (or consisting of) a secondary amine and an organic acid. Just about any secondary amine or organic acid can be used, as long as the molecules are not too big (e.g., less than or equal to 10 carbon atoms, preferably less than 10 carbon atoms). Possible acids include formic, acetic, propionic, and higher acids, as well as diacids such as succinic, maleic, and malic acids, as well as combinations comprising at least one of the foregoing acids. Possible secondary amines include dimethyl, diethyl, and higher amines, mixed amines (such as methyl ethyl and ethyl butyl), plus cyclic amines (such as piperidine, piperazine, and morpholine), as well as combinations comprising at least one of the foregoing amines.

Methacrolein can be converted directly to methyl methacrylate by a reaction known as oxidative esterification, that is, esterification in the presence of oxygen. Typically, esterification is a reaction between an alcohol, methanol in this case, and an acid, which would be methacrylic acid in this case. With oxidative esterification, the aldehyde (e.g., methacrolein) is used directly in the reaction without prior oxidation to the acid. The mechanism of oxidative esterification is not totally clear, but it does not appear to involve sequential oxidation of the methacrolein followed by esterification.

Various catalysts can be used for this reaction, such as a palladium/lead catalyst. The catalyst can comprise various supports such as silica, polystyrene/divinyl benzene, and others. In this catalyst, the lead deposition is carefully controlled to form a very pure Pd₃Pd intermetallic compound, with little or none of either free palladium or free lead present (e.g., with less than 5% of the lead being free lead and/or less than 5% of the palladium being free palladium).

### EXAMPLES

### Example 1

In one example, a rhodium catalyst can be prepared by dissolving 0.0588 grams (g) rhodium dicarbonyl saliclaldoximate in 10 milliliters (ml) of toluene, and then adding 0.0524 ml triphenyl phosphite. This reaction forms the complex salicylaldoximatocarbonyltriphenylphosphiterhodium in toluene.

A 1 ml portion of this catalyst complex solution can be put in an autoclave with 99 ml of additional toluene. After nitrogen purging, the autoclave is pressured to 550 pounds per square inch gauge (psig) with ethylene, then to 1200 psig with a 1:1 gas (a mixture of hydrogen and carbon monoxide). It is then heated to about 90° C. for 10 hours. As the pressure falls due to the reaction, additional 1:1 gas is added to maintain the pressure. After 10 hours, the autoclave is cooled and vented. Propionaldehyde is obtained at a selectivity of almost 99%, with the remainder being mostly diethyl ketone.

### Example 2

In one example, 104.4 g of propionaldehyde, 2 g of propionic acid, and 98 g of 30% aqueous formaldehyde are mixed in a vessel, with 5.8 g of di-n-butylamine added with cooling. Once the amine addition is completed, the reactor is heated to about 100° C. for about one hour. Upon cooling, the reaction mixture forms two phases, one organic and one aqueous. The organic phase contains over 90% methacrolein.

### Example 3

50.1 g of methacrolein is added to the reactor, along with 25.2 g of methanol (for a molar ratio of methanol:methacrolein of about 1.1). Roughly 1 g of catalyst (e.g., 3% palladium and 2% lead on silica) is added to the solution. A stirrer is turned on, and the solution is heated to about 50° C. Oxygen flow is begun at about 6 milliliters per minute (ml/min). The reactor is open to atmospheric pressure. The reaction is continued for about 4 hours. This results in methacrolein conversion of about 50%, with selectivity to methyl methacrylate of about 90%.

Set forth below are some embodiments of the method disclosed herein.

### Embodiment 1

A process for forming methyl methacrylate, comprising: reacting ethylene, carbon monoxide, and hydrogen, in the presence of a first catalyst comprising a metal carbonyl; removing a first reaction product comprising propionaldehyde; reacting the first reaction product with formaldehyde; removing a second reaction product comprising methacrolein; reacting the second reaction product with oxygen and methanol in the presence of a second catalyst to form a third reaction product comprising methyl methacrylate.

### Embodiment 2

The process of Embodiment 1, wherein the first catalyst is a homogeneous catalyst and comprises at least one of cobalt, rhodium, iridium, and ruthenium in combination with a biphyllic ligand comprising at least one of phosphorus, arsenic, and bismuth.

### Embodiment 3

The process of Embodiments 1 or 2, wherein the reacting ethylene, carbon monoxide, and hydrogen further comprises a solvent.

### Embodiment 4

The process of any of Embodiments 1-3, wherein the metal carbonyl is in the presence of a ligand.

### Embodiment 5

The process of any of Embodiments 1-4, wherein the first reaction product is further reacted with a secondary amine and an organic acid.

### Embodiment 6

The process of Embodiment 5, wherein the secondary amine comprises di-2-ethyl hexylamine, diphenylamine, dicyclohexylamine, dipropylamine, methylbutylamine, ethylbutylamine, diisooctylamine, piperidine, pyrrolidine, piperazine, morpholine, and combinations thereof.

### Embodiment 7

The process of any of Embodiments 5-6, wherein the organic acid comprises formic acid, oxalic acid, maleic acid, acetylene, dicarboxylic acid, acetic acid, propionic acid, n- or i-butanoic acid, malonic acid, glutaric acid, succinic acid, tartaric acid, adipic acid, hydroxy succinic acid, salicylic acid, 2-ethylhexanoic acid, and combinations thereof.

### Embodiment 8

The process of any of Embodiments 1-7, wherein the reacting of the ethylene, the carbon monoxide, and the hydrogen, in the presence of the first catalyst is at a reaction temperature of 50° C. to 200° C.

### Embodiment 9

The process of any of Embodiments 5-8, wherein the reacting of the ethylene, the carbon monoxide, and the hydrogen, in the presence of the first catalyst is at a reaction temperature of 70° C. to 120° C.

### Embodiment 10

The process of any of Embodiments 5-9, wherein the reacting of the ethylene, the carbon monoxide, and the hydrogen, in the presence of the first catalyst is at an autogeneous pressure of 101.3 kPa to 303.9 kPa.

### Embodiment 11

The process of any of Embodiments 1-10, wherein the reacting of the ethylene, the carbon monoxide, and the hydrogen, in the presence of the first catalyst is at a reaction pressure of 790 kPa to 20785 kPa.

### Embodiment 12

The process of any of Embodiments 1-11, further comprising providing the formaldehyde in a molar ratio of 1.5:1 to 1:1 to the propionaldehyde.

### Embodiment 13

The process of any of Embodiments 1-12, wherein the second catalyst comprises a heterogeneous catalyst comprising: at least one of palladium, rhodium, and ruthenium; and at least one of lead, mercury, thallium, gold, copper, silver, cadmium, zinc, indium, tin, antimony, and bismuth.

### Embodiment 14

The process of any of Embodiments 1-13, wherein the second catalyst comprises palladium and gold.

### Embodiment 15

The process of any of Embodiments 1-14, wherein the second catalyst comprises palladium and lead.

### Embodiment 16

The process of Embodiment 15, wherein a molecular ratio of palladium to lead is 3 to 1.

### Embodiment 17

A process for forming methyl methacrylate, comprising: reacting ethylene with carbon monoxide to form propionaldehyde; reacting the propionaldehyde with formaldehyde to form methacrolein; and reacting the methacrolein with methanol and oxygen to form the methyl methacrylate.

### Embodiment 18

The process of Embodiment 17, wherein the propionaldehyde is produced in a yield greater than about 95 percent.

### Embodiment 19

The process of any of Embodiments 17-18, wherein the reacting of the methacrolein with methanol and oxygen further comprises the presence of a catalyst.

### Embodiment 20

The process of any of Embodiments 17-19, wherein the reacting the methacrolein with methanol and oxygen further comprises the presence of a catalyst comprising palladium.

### Embodiment 21

The process of any of Embodiments 17-20, wherein the reacting of the methacrolein with methanol and produces the methyl methacrylate at a conversion rate of greater than about 95 percent of the methacrolein.

### Embodiment 22

The process of any of Embodiments 1-21, wherein the methacrolein is produced in a yield greater than about 95 percent.

All ranges disclosed herein are inclusive of the endpoints, and the endpoints are independently combinable with each other (e.g., ranges of “up to 25 wt %, or, more specifically, 5 wt % to 20 wt %”, is inclusive of the endpoints and all intermediate values of the ranges of “5 wt % to 25 wt %,” etc.). “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements can be combined in any suitable manner in the various embodiments. Unless defined otherwise, technical and scientific terms used herein have the same meaning as is commonly understood by one of skill in the art to which this invention belongs.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

While typical embodiments have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

What is claimed is:

1. A process for forming methyl methacrylate, comprising:
reacting ethylene, carbon monoxide, and hydrogen, in the presence of a first catalyst comprising a metal carbonyl;
removing a first reaction product comprising propionaldehyde;
reacting the first reaction product with formaldehyde;
removing a second reaction product comprising methacrolein;
reacting the second reaction product with oxygen and methanol in the presence of a second catalyst to form a third reaction product comprising methyl methacrylate.

2. The process of claim 1, wherein the first catalyst is a homogeneous catalyst and comprises at least one of cobalt, rhodium, iridium, and ruthenium in combination with a biphyllic ligand comprising at least one of phosphorus, arsenic, and bismuth.

3. The process of claim 1, wherein the reacting ethylene, carbon monoxide, and hydrogen further comprises a solvent.

4. The process of claim 1, wherein the metal carbonyl is in the presence of a ligand.

5. The process of claim 1, wherein the first reaction product is further reacted with a secondary amine and an organic acid.

6. The process of claim 5, wherein the secondary amine comprises di-2-ethyl hexylamine, diphenylamine, dicyclohexylamine, dipropylamine, methylbutylamine, ethylbutylamine, diisooctylamine, piperidine, pyrrolidine, piperazine, morpholine, and combinations thereof.

7. The process of claim 5, wherein the organic acid comprises formic acid, oxalic acid, maleic acid, acetylene, dicarboxylic acid, acetic acid, propionic acid, n- or i-butanoic acid, malonic acid, glutaric acid, succinic acid, tartaric acid, adipic acid, hydroxy succinic acid, salicylic acid, 2-ethylhexanoic acid, and combinations thereof.

8. The process of claim 1, wherein the reacting of the ethylene, the carbon monoxide, and the hydrogen, in the presence of the first catalyst is at a reaction temperature of 50° C. to 200° C.

9. The process of claim 8, wherein the reacting of the ethylene, the carbon monoxide, and the hydrogen, in the presence of the first catalyst is at a reaction temperature of 70° C. to 120° C.

10. The process of claim 5, wherein the reacting of the ethylene, the carbon monoxide, and the hydrogen, in the presence of the first catalyst is at an autogeneous pressure of 101.3 kPa to 303.9 kPa.

11. The process of claim 1, wherein the reacting of the ethylene, the carbon monoxide, and the hydrogen, in the presence of the first catalyst is at a reaction pressure of 790 kPa to 20785 kPa.

12. The process of claim 1, further comprising providing the formaldehyde in a molar ratio of 1.5:1 to 1:1 to the propionaldehyde.

13. The process of claim 1, wherein the second catalyst comprises a heterogeneous catalyst comprising
at least one of palladium, rhodium, and ruthenium; and
at least one of lead, mercury, thallium, gold, copper, silver, cadmium, zinc, indium, tin, antimony, and bismuth.

14. The process of claim 1, wherein the second catalyst comprises palladium and gold.

15. The process of claim 1, wherein the second catalyst comprises palladium and lead.

16. The process of claim 15, wherein a molecular ratio of palladium to lead is 3 to 1.
